(19) United States
(12) Patent Application Publication
(10) Pub. No.: US 2025/0226101 A1
(43) Pub. Date: Jul. 10, 2025

(54) METHOD AND DEVICE FOR DETECTING SIDE EFFECT DRUG AND PROVIDING OPTIMIZED DRUGS USING PERSONALIZED SIDE EFFECT DATA

(71) Applicants: Min Gyu Kang, Cheongju-si (KR); CHUNGBUK NATIONAL UNIVERSITY HOSPITAL, Cheongju-si (KR)

(72) Inventor: Min Gyu KANG, Cheongju-si (KR)

(73) Assignees: Min Gyu Kang, Cheongju-si (KR); CHUNGBUK NATIONAL UNIVERSITY HOSPITAL, Cheongju-si (KR)

(21) Appl. No.: 19/091,707

(22) Filed: Mar. 26, 2025

Related U.S. Application Data
(63) Continuation of application No. PCT/KR2023/009195, filed on Jun. 30, 2023.

(30) Foreign Application Priority Data
Sep. 27, 2022 (KR) 10-2022-0122884

(51) Int. Cl. G16H 50/20; G16H 10/60; G16H 20/10; G16H 70/40

(52) U.S. Cl. CPC G16H 50/20; G16H 10/60; G16H 20/10; G16H 70/40

(57) ABSTRACT

The present disclosure provides a method for detecting an adverse reaction-inducing drug and providing an optimized drug using individualized adverse reaction data. The method includes receiving prescription information from a user, receiving adverse reaction information from the user, extracting a specific drug inducing an adverse reaction by analyzing a possibility of the adverse reaction occurring based on drugs included in the prescription information and the adverse reaction information using an individualized adverse reaction database and determining an alternative drug, generating optimized prescription information based on the alternative drug and providing the optimized prescription information to the user, and collecting feedback information after drug administration based on the optimized prescription information from the user to update the individualized adverse reaction database.

### CROSS-REFERENCE TO RELATED APPLICATIONS

The present application is a continuation of International Patent Application No. PCT/KR2023/009195, filed on Jun. 30, 2023, which is based upon and claims the benefit of priority to Korean Patent Application No. 10-2022-0122884 filed on Sep. 27, 2022. The disclosures of the above-listed applications are hereby incorporated by reference herein in their entirety.

### BACKGROUND

The present disclosure relates to techniques for detecting a drug having a risk of inducing an adverse reaction and recommending and/or providing an optimized drug. More specifically, the disclosure relates to a method and a device for detecting an adverse reaction-inducing drug and providing an optimized drug using individualized adverse reaction data.

In recent years, the frequency and amount of drug consumption have been increasing rapidly due to the aging global population. The number of adverse reactions is also increasing rapidly, threatening patient safety. Therefore, analyzing and identifying new or severe adverse reactions of marketed drugs at an early stage has emerged as a critical issue and to achieve this, collecting data from drug users is essential.

Additionally, it is important to utilize the collected data to predict adverse reactions of drugs that users may take in the future and to provide drugs with a lower risk of adverse reactions.

### SUMMARY

Embodiments of the present disclosure provide a method and a device for detecting an adverse reaction-inducing drug and providing an optimized drug using individualized adverse reaction data.

However, problems to be solved by the present disclosure may not be limited to the above-described problems. Although not described herein, other problems to be solved by the present disclosure may be clearly understood by those skilled in the art from the following description.

According to an embodiment, a method performed by a server for detecting an adverse reaction-inducing drug and providing an optimized drug using individualized adverse reaction data, includes receiving prescription information from a user, receiving adverse reaction information from the user, extracting a specific drug inducing an adverse reaction by analyzing a possibility of the adverse reaction occurring based on drugs included in the prescription information and the adverse reaction information using an individualized adverse reaction database and determining an alternative drug, generating optimized prescription information based on the alternative drug and providing the optimized prescription information to the user; and collecting feedback information after drug administration based on the optimized prescription information from the user to update the individualized adverse reaction database.

In an embodiment, the extracting of the specific drug inducing the adverse reaction and determining of the alternative drug may include extracting the specific drug inducing the adverse reaction by analyzing the possibility of the adverse reaction occurring based on the drugs included in the prescription information and the adverse reaction information and determining an alternative drug, using a learning model trained based on the individualized adverse reaction database.

In an embodiment, the extracting of the specific drug inducing the adverse reaction and determining of the alternative drug may include calculating a probability of the adverse reaction by analyzing the possibility of the adverse reaction occurring based on the drugs included in the prescription information and the adverse reaction information using the individualized adverse reaction database according to a statistical methodology, and extracting the specific drug inducing the adverse reaction and determining the alternative drug based on whether the possibility of the adverse reaction occurring exceeds a predetermined threshold.

In an embodiment, the extracting of the specific drug inducing the adverse reaction and determining of the alternative drug may include receiving a response from the user to a question relating to the possibility of the adverse reaction occurring based on the drugs included in the prescription information and the adverse reaction information, and determining, based on the response, the possibility of the adverse reaction occurring based on the drugs included in the prescription information and the adverse reaction information.

In an embodiment, the method may further include building the individualized adverse reaction database using individualized adverse reaction data obtained based on at least one of user information, the prescription information, and the adverse reaction information, and generating, the learning model for extracting an adverse reaction-inducing drug and determining an alternative drug through machine learning based on the individualized adverse reaction database.

In an embodiment, the learning model may be further trained to extract the adverse reaction-inducing drug and determine the alternative drug through the machine learning based on the individualized adverse reaction database updated with feedback information collected after drug administration based on the optimized prescription information,

In an embodiment, the extracting of the specific drug inducing the adverse reaction and determining of the alternative drug may include extracting the specific drug inducing the adverse reaction and determining the alternative drug by further using the individualized adverse reaction database and external databases.

In an embodiment, the method may further include providing at least one of the adverse reaction information, the specific drug inducing the adverse reaction, the alternative drug, the optimized prescription information, or the feedback information to an external server.

According to an embodiment, a device for providing a method for detecting an adverse reaction-inducing drug and providing an optimized drug using individualized adverse reaction data includes an information reception unit configured to receive prescription information from a user and receive adverse reaction information from the user, an analysis unit configured to extract a specific drug inducing an adverse reaction by analyzing a possibility of the adverse reaction occurring based on drugs included in the prescription information and the adverse reaction information using an individualized adverse reaction database and determine an alternative drug, an information provision unit configured to generate optimized prescription information based on the alternative drug and provide the optimized prescription information to the user, and an update unit configured to collect feedback information after drug administration based on the optimized prescription information from the user to update the individualized adverse reaction database.

In addition, a computer program stored on a computer-readable recording medium for execution to implement the present disclosure may be further provided.

In addition, a computer-readable recording medium for recording a computer program for executing a method for implementing the present disclosure may be further provided.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a block diagram schematically illustrating a device that performs a method for detecting adverse reaction-inducing drugs using personalized adverse reaction data and providing optimized drugs according to an embodiment of the present disclosure;

FIG. 2 is a block diagram schematically illustrating a device that performs a method for detecting adverse reaction-inducing drugs using personalized adverse reaction data and providing optimized drugs according to an embodiment of the present disclosure;

FIG. 3 is a flowchart schematically illustrating a method for detecting adverse reaction-inducing drugs using personalized adverse reaction data and providing optimized drugs according to an embodiment of the present disclosure; and

FIGS. 4 to 10 illustrate examples of algorithms for analyzing a causal relationship between drugs and adverse reactions or the possibility of adverse reactions occurring according to an embodiment of the present disclosure.

### DETAILED DESCRIPTION

Advantages and features of the present disclosure and methods for achieving them will be apparent with reference to embodiments described below in detail in conjunction with the accompanying drawings. However, the inventive concept is not limited to the embodiments disclosed below, but may be implemented in various forms, and these embodiments are to make the disclosure of the inventive concept complete, and are provided so that this disclosure will be thorough and complete and will fully convey the scope of the invention to those of ordinary skill in the art, which is to be defined only by the scope of the claims.

The terminology used herein is for the purpose of describing particular embodiments only and is not intended to be limiting of the inventive concept. The singular expressions include plural expressions unless the context clearly dictates otherwise. In this specification, the terms “comprises” and/or “comprising” are intended to specify the presence of stated elements, but do not preclude the presence or addition of elements. Like reference numerals refer to like elements throughout the specification, and “and/or” includes each and all combinations of one or more of the mentioned elements. Although “first”, “second”, and the like are used to describe various components, these components are of course not limited by these terms. These terms are only used to distinguish one component from another. Thus, a first element discussed below could be termed a second element without departing from the teachings of the inventive concept.

Unless otherwise defined, all terms (including technical and scientific terms) used herein have the same meaning as commonly understood by one of ordinary skill in the art to which this invention belongs. It will be further understood that terms such as those defined in commonly used dictionaries will not be interpreted in an idealized or overly formal sense unless expressly so defined herein.

Like reference numerals refer to like elements throughout the specification. The present disclosure does not describe all elements of the embodiments, and general content in the technical field to which the present disclosure pertains or overlapping content between embodiments are omitted. Terms such as “unit”, “module”, “member”, and “block” may be embodied as hardware or software. According to forms, a plurality of “unit”, “module”, “member”, and “block” may be implemented as a single component or a single “unit”, “module”, “member”, and “block” may include a plurality of components.

It will be understood that when an element is referred to as being “connected” to another element, it may be directly or indirectly connected to the other element, wherein the indirect connection includes “connection via a wireless communication network”. Also, when a part “includes” or “comprises” an element, unless there is a particular description contrary thereto, the part may further include other elements, not excluding the other elements.

Throughout the specification, a certain member being located “on” another member includes not only a case where the certain member is in contact with another member, but also a case where another member exists between two members.

In each step, identification symbols are used for convenience of description and the identification symbols do not describe the order of the steps and the steps may be performed in a different order from the described order unless the context clearly indicates a specific order.

Hereinafter, the operating principle and embodiments of the present disclosure will be described with reference to the attached drawings.

Previously, there was a problem in that no device existed to store and process an individual patient's drug experience. However, although there is publicly available data on a possibility to induce adverse reactions for each drug, the data generally represents the overall probability of occurrence rather than individual-specific risks. However, it does not always mean that there is a possibility of the event that an individual with certain characteristics, including genetics, will experience adverse reactions “B” after taking drug “A”.

For example, if individual “X” takes drug “A” and monitors to see if adverse reactions “B” occurs or does not occur, the posterior possibility that individual “X” will experience adverse reaction “B” after taking drug “A” may be adjusted based on that event.

By monitoring the occurrence of adverse reaction in individuals, an individualized database for probabilities of adverse reaction occurring may be established.

Hereinafter, embodiments of the present disclosure will be described in detail.

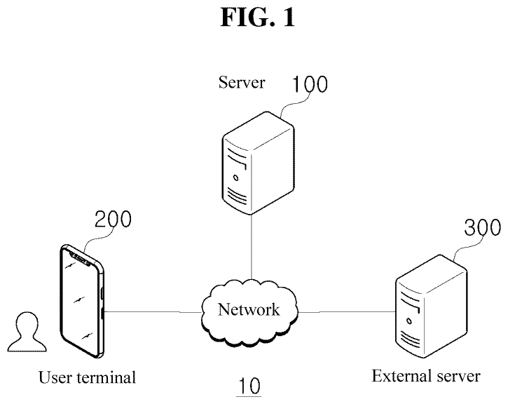

FIG. 1 schematically illustrates a system in which a method of detecting adverse reaction-inducing drugs and providing optimized drugs using individualized adverse reactions data is performed, according to an embodiment of the present disclosure.

Referring to FIG. 1, a system 10 in which a method of detecting adverse reaction (AR)-inducing drugs and providing optimized drugs using individualized adverse reaction data according to one embodiment of the present disclosure is performed may include a server 100 and at least one user terminal 200. In this case, the system 10 shown in FIG. 1 is an example only, and may include fewer or more components than the components shown in FIG. 1.

The server 100 may be a computing device that performs a method of detecting AR-inducing drugs and providing optimized drugs using individualized ADR data according to an embodiment of the present disclosure. Further, the server 100 may include any of a variety of devices capable of performing computational processing necessary to perform the the method according to an embodiment of the present disclosure and providing results thereof to a user. Additionally, the server 100 may include a computer system and computer software (web server program) that derive and provide task results in response to task execution requests from clients or other web servers. In addition to the web server program described above, the server 100 may include a set of application programs that operate on the web server, or various databases built into the device. The server may include, for example, a computer, a server device, and a portable terminal, or it may take the form of any one of these.

Here, the computer may include, for example, a notebook, desktop, laptop, tablet PC, slate PC, or the like, which is equipped with a web browser.

The server device is a server that processes information by communicating with an external device, and may include an application server, a computing server, a database server, a file server, a game server, a mail server, a proxy server, a web server and/or the like.

The portable terminal may be, for example, a wireless communication device that is portable and mobile and may include all types of handheld-type wireless communication devices, such as PCS (Personal Communication System), GSM (Global System for Mobile communications), PDC (Personal Digital Cellular), PHS (Personal Handyphone System), PDA (Personal Digital Assistant), IMT (International Mobile Telecommunication)-2000, CDMA (Code Division Multiple Access)-2000, W-CDMA (W-Code Division Multiple Access), or WiBro (Wireless Broadband Internet) terminals, and wearable devices such as watches, rings, bracelets, anklets, necklaces, glasses, contact lenses, or head-mounted-device (HMD), or the like.

The server 100 may provide a service for detecting AR-inducing drugs using individualized ADR data and providing optimized drugs to at least one or more user terminals 200 connected via a network. A detailed description thereof will be provided later.

The user terminal 200 may be a computing device capable of connecting to the server 100 via a network, inputting information, and outputting results thereof. In an embodiment, the user terminal 200 may include various computing devices equipped with a communication device such as a communication modem for performing communication with various devices or wired/wireless networks, a memory for storing various programs and data, and a microprocessor for executing programs to perform computations and control operations. For example, the user terminal may be a computer or a portable device, as described above.

In an embodiment, a patient may access a platform providing a service for detecting AR-inducing drugs and providing optimized drugs using individualized ADR data through the user terminal 200 and input information such as prescription information prescribed by a doctor, adverse reactions after taking drug, and effects of the drug via the platform. The information input into the platform may be transmitted to the server 100, and subsequently, the patient may receive results (AR-inducing drugs, optimized drugs etc.) corresponding to the input information (e.g., prescription information, optimized drug information, effects information etc.) from the server 100 and display them on the platform screen via the user terminal 200.

Additionally, according to an embodiment, the method for detecting AR-inducing drugs using individualized adverse reaction data and providing optimized drugs according to an embodiment of the present disclosure may be performed in association with doctors, pharmacists, hospitals, pharmacies, and delivery services. In other words, the method may also be linked to at least one external server 300 or other platforms.

In an embodiment, the external server 300 may implement a platform that provides services for individualized drug recommendations and adverse reaction prevention, allowing doctors, pharmacists, hospitals, and pharmacies to utilize the platform. Furthermore, the platform may be used not only by medical institutions or professionals such as doctors, pharmacists, and pharmacies but also by specific individuals such as guardians of a patient or those who have been granted authority, including companies. For example, a professional terminal (e.g., a doctor or a pharmacist) may access the platform provided by the external server 300 and obtain information on previously prescribed drugs, drugs that have induced adverse reactions, and optimized drugs for the patient. In this case, the information on the prescribed drugs, drugs that have induced adverse reactions, and optimized drugs for the patient may have been received from the platform that detects AR-inducing drugs using individualized adverse reaction data and provides optimized drugs (i.e., server 100). Based on this information, a professional terminal (e.g., a doctor or a pharmacist) may predict potential adverse reactions during the prescription or dispensing stage according to the type of drug and prescribe or dispense drugs with lower adverse reaction risks for each patient. Additionally, a professional terminal (e.g., a doctor or a pharmacist) may receive individualized drug recommendations for patients through the platform. Moreover, in some embodiments, the external server 300 may be linked to or pass through drug reaction management specialized institutions' servers, such as the DUR server of the Health Insurance Review and Assessment Service or the Korea Institute of Drug Safety & Risk Management, to implement the platform and provide services. Alternatively, in some embodiments, the external server 300 may be linked to or pass through external EMR (Electronic Medical Record) companies, or it may be integrated and operated with the EMR system of a general hospital.

In an embodiment, the external server 300 may implement a platform that provides an AI-based remote drug guidance service, allowing pharmacists and pharmacies to use it. For example, a professional terminal (e.g., a pharmacist or a pharmacy) may prescribe drugs with lower adverse reaction risks to patients and receive individualized drug recommendations for each patient through the platform, as mentioned earlier. In this case, an AI pharmacist may be utilized to remotely guide drug usage or provide counseling on prescribed drugs.

In another embodiment, the external server 300 may implement a drug delivery platform, allowing the delivery service to use it. For example, a delivery terminal (e.g., a delivery service company, a courier personnel, etc.) may receive information on prescribed drugs for a patient from a professional terminal (e.g., a doctor or a pharmacist) via the drug delivery platform provided by the external server 300. The delivery personnel may then deliver the prescribed drugs (i.e., the prescription) or drugs dispensed based on the prescription to a corresponding patient.

The aforementioned platforms may each be implemented on the external servers 300 and provided as services, and the external servers 300 may be different from one another. In some embodiments, the aforementioned platforms may be integrated into a single platform and provided as a service. In this case, the platforms may be implemented and provided on the server 100. That is, the described embodiments are merely examples and are not limited to these. The embodiments may be modified and applied in various ways.

Meanwhile, the network may transmit and receive a variety of information among the server 100, at least one user terminal 200, and the external server 300. The network (not shown) may use various types of communication networks, for example, wireless communication schemes including WLAN (Wireless LAN), Wi-Fi, Wibro, Wimax, HSDPA (High Speed Downlink Packet Access), and the like, or wired communication schemes including Ethernet, xDSL (ADSL, VDSL), HFC (Hybrid Fiber Coax), FTTC (Fiber to The Curb), FTTH (Fiber To The Home) and the like.

On the other hand, the network is not limited to the communication schemes presented above, and may include all types of communication schemes widely known or to be developed in the future in addition to the above-described communication schemes.

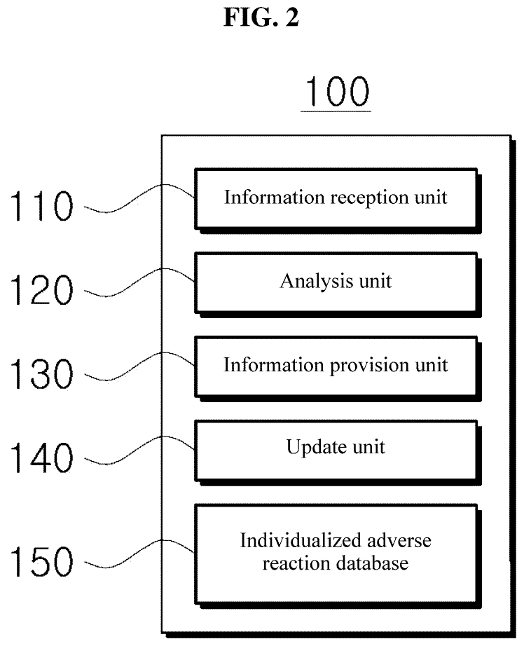

FIG. 2 is a block diagram schematically illustrating a device that performs a method for detecting AR-inducing drugs using individualized adverse reaction data and providing optimized drugs according to an embodiment of the present disclosure.

The device 100 that performs the method for detecting AR-inducing drugs using individualized adverse reaction data and providing optimized drugs data according to an embodiment of the present disclosure, as disclosed in FIG. 2, corresponds to the server 100 of FIG. 1 described above. That is, the device 100 disclosed in FIG. 2 may include all types of devices capable of performing computational processing and providing results to users, as described above. For example, the device 100 disclosed in FIG. 2 may include a computer, a server device, and a portable terminal, or it may be in the form of any one of these.

Referring to FIG. 2, the device 100 that performs the method for detecting AR-inducing drugs using individualized adverse reaction data and providing optimized drugs data according to an embodiment of the present disclosure may include an information reception unit 110, an analysis unit 120, an information provision unit 130, an update unit 140, and an individualized adverse reaction database 150. The components shown in FIG. 2 are not essential for implementing the device 100 according to the present disclosure, meaning that the device 100 described in this specification may have more or fewer components than those shown in FIG. 2.

The information reception unit 110 may receive prescription information from the user terminal 200. Additionally, the information reception unit 110 may receive adverse reaction information from the user terminal 200.

Here, the prescription information may include patient-related details (e.g., name, age, gender, resident registration number, etc.), medical institution information (e.g., name, address, phone number, etc.), prescribing physician information, disease information, and prescribed drug details (e.g., prescribed drug (drug) name, dosage, duration, frequency, usage instructions, etc.). The adverse reaction information may include condition details following drug administration, adverse reaction symptoms, and drug efficacy or the like. Additionally, the adverse reaction information may include condition details following drug administration, adverse reaction symptoms, and drug efficacy or the like.

Furthermore, in some embodiments, the information reception unit 110 may receive prescription information from a physician's EMR or a pharmacist's terminal via an external network. That is, the user terminal may not only be a device used by a patient but also a device used by a physician or pharmacist to input various types of information.

Moreover, in some embodiments, the information reception unit 110 may transmit prescription information to the Health Insurance Review and Assessment Service (HIRA) DUR system when a prescription is issued at a hospital. In this case, the system (e.g., a DUR-related module) may transmit the prescription information and also transmit adverse reaction risk information. In other words, the Health Insurance Review and Assessment Service, which has received the prescription information, may transmit the information back to the information reception unit 110.

The analysis unit 120 may analyze causal relationships between drugs included in the prescription information and the adverse reaction information or the possibility of adverse reactions occurring based on the individualized adverse reaction database 150 to extract specific drugs that induce adverse reactions and determine alternative drugs.

In this case, the analysis unit 120 may analyze causal relationships between the drugs included in the prescription information and the adverse reaction information and the possibility of adverse reactions occurring to extract specific drugs that induce or are highly likely to induce adverse reactions by using not only the individualized adverse reaction database 150 but also external databases and determine alternative drugs. Since the individualized adverse reaction database 150 may not have sufficient data in its initial stage, external databases may be utilized. For example, external databases may include databases built by collecting, assessing and organizing pharmaceutical-related data from institutions such as the Ministry of Food and Drug Safety (MFDS), HIRA, the Ministry of Health and Welfare, the U.S. FDA, and the European Medicines Agency (EMA). Alternatively, external databases may include databases established by agencies such as the MFDS, the Korea Institute of Drug Safety & Risk Management, the U.S. FDA, and the EMA, which collect and evaluate adverse reaction data. In another example, external databases may reference individualized adverse reaction records input into the “My drug” system of the Health Insurance Review and Assessment Service. Additionally, in another example, external databases may include platforms such as the My Healthway platform or other third-party platforms designed for patient information sharing.

Furthermore, in some embodiments, the analysis unit 120 may analyze whether factors, such as a patient's demographic characteristics (e.g., age, gender) and comorbidity information, influence the risk of adverse reactions, in addition to the individualized adverse reaction data. Based on the analysis results, the analysis unit 120 may extract specific drugs that influence occurrence of adverse reactions and determine alternative drugs.

The information provision unit 130 may generate prescription information optimized based on the alternative drugs and transmit the prescription information to the user terminal 200. Thus, since AR-inducing drugs are extracted among drugs included in the prescription, the information provision unit 130 may re-prescribe an optimized prescription to the user (i.e., the patient) by including alternative drugs instead of the adverse reaction-inducing drugs. Here, the alternative drugs may include drugs that are expected to have the same or similar effects while simultaneously having a lower possibility of inducing adverse reactions.

The update unit 140 may collect feedback information on drug administration based on the optimized prescription information from the user terminal 200 and update the individualized adverse reaction database 150. Here, the feedback information may include whether adverse reactions occurred, symptoms, severity, and temporal relationships.

The individualized adverse reaction database 150 may be composed of a collection of individualized adverse reaction data acquired based on user information (i.e., patient information), prescription information, and adverse reaction information (including both publicly known adverse reaction information and personally known adverse reaction information). In other words, the individualized adverse reaction database 150 may be composed of a collection of individualized adverse reaction data for each drug, which is acquired by updating the possibility of adverse reactions occurring for specific drugs using a separate algorithm based on adverse reaction information occurred after drug administration.

As described above, user information (i.e., patient information or disease information) may include details such as name, age, gender, resident registration number, address, or the like. The prescription information may include patient details (e.g., name, age, gender, resident registration number, etc.), medical institution information (e.g., name, address, phone number, etc.), prescribing physician information, disease information, and prescribed drug details (e.g., drug (drug) name, dosage, duration, frequency, usage instructions, etc.). The adverse reaction information may include condition details following drug administration, adverse reaction symptoms, and drug efficacy or the like. Additionally, the adverse reaction information may further include updated information on previously evaluated, identified, or experienced adverse reactions before using the platform. That is, through the use of the platform according to an embodiment of the present disclosure, previously evaluated, identified, or experienced adverse reaction information may be updated, thereby making the adverse reaction information more accurate.

In an embodiment, the individualized adverse reaction database 150 may be built based on each patient's drug adverse reaction history and the probability of adverse reactions occurring. Alternatively, the individualized adverse reaction database 150 may be built by allowing patients to input symptom changes (e.g., effects, adverse reactions, etc.) through the user terminal 200 after drug administration. Additionally, the individualized adverse reaction database 150 may maintain the individualized adverse reaction data in an optimized state in such a way to calculate the probability of adverse reactions based on drug efficacy and adverse reaction symptoms and update the possibility of adverse reactions occurring. For example, the probability of adverse reactions may be calculated using statistical methodologies based on drug efficacy and adverse reaction symptoms and the individualized adverse reaction database 150 may then be updated based on whether the calculated probability exceeds a predefined threshold.

Additionally, although not shown in FIG. 2, the device 100 that performs the method for detecting adverse reaction-inducing drugs and providing optimized drugs using individualized adverse reaction data according to an embodiment of the present disclosure may further include a memory unit and a control unit.

The memory unit may store data supporting various functions of the host device and programs for operation of the control unit, may store input/output data (e.g., music files, still images, videos, and more), and may store a number of applications (application programs or applications) running on the host device, and data and instructions for operation of the host device. At least some of these application programs may be downloaded from an external server through wireless communication.

The memory unit may include at least one type of storage medium among a flash memory type, a hard disk type, a solid state disk type, an silicon disk drive type (SDD) type, a multimedia card micro type, a card type memory (e.g., SD or XD memory), a random access memory (RAM), a static random access memory (SRAM), a read-only memory (ROM), an electrically erasable programmable read-only memory (EEPROM), a programmable read-only memory (PROM), a magnetic memory, a magnetic disk, and an optical disk. Furthermore, the memory unit may be a database that is separate from the host device but connected thereto in a wired or wireless manner.

The control unit may be implemented with a memory that stores data for an algorithm or program reproducing the algorithm for controlling the operations of components within the device, and at least one processor (not shown) that performs the aforementioned operations using the data stored in the memory. In this case, the memory and the processor may be implemented as separate chips. Alternatively, the memory and the processor may be implemented on a single chip.

Further, the control unit may control any one or a plurality of the above-described components in combination to implement various embodiments of the present disclosure described below in the device.

At least one component may be added or deleted in response to the performance of the components shown in FIG. 2. Additionally, it will be easily understood by those skilled in the art that the mutual positions of the components may be changed in response to the performance or structure of a system.

Meanwhile, each of components shown in FIG. 2 may refer to a software and/or hardware component such as Field Programmable Gate Array (FPGA) and Application Specific Integrated Circuit (ASIC).

In the following, a method for detecting AR-inducing drugs using individualized adverse reaction data and providing optimized drugs, performed in the device 100 according to an embodiment of the present disclosure described above, will be described in detail.

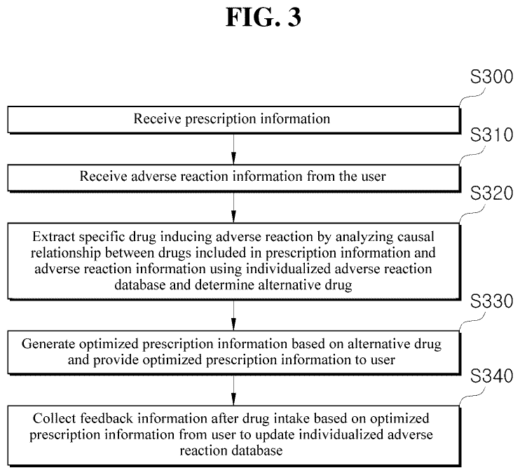

FIG. 3 is a flowchart schematically illustrating a method for detecting AR-inducing drugs using individualized adverse reaction data and providing optimized drugs according to an embodiment of the present disclosure.

The method of FIG. 3 may be performed by the device 100 disclosed in FIG. 2. Here, the device 100 disclosed in FIG. 2 may be a computing device (e.g., a computer, a server device, a handheld terminal, etc.), in which case the method of FIG. 3 may be performed by the computing device (more specifically, a server).

Referring to FIG. 3, the information reception unit 110 may receive prescription information from a user (S300).

As described above, the prescription information may include patient details (e.g., name, age, gender, resident registration number, etc.), medical institution information (e.g., name, address, phone number, etc.), prescribing physician information, disease information, and prescribed drug details (e.g., drug (drug) name, dosage, duration, frequency, usage instructions, etc.).

For example, a user (i.e., a patient) may input prescription information including drugs prescribed to the user via a platform or application on the user terminal 200. The inputted prescription information may be transmitted from the user terminal 200 to the information reception unit 110.

Here, a platform or an application is implemented to provide a service for detecting AR-inducing drugs and providing optimized drugs using individualized adverse reaction data and may be downloaded to the user terminal 200 from the server 100 and installed on the user terminal 200. The user may execute the platform or the application installed on the user terminal 200 to receive an interface for inputting prescription information. For example, the user may execute the platform or the application installed on the user terminal 200 and then input prescription information by photographing the prescription information via a camera, and the photographed prescription information may be transmitted to the information reception unit 110. Further, in accordance with an embodiment, when a QR code included in the prescription information is recognized by executing the platform or the application installed on the user terminal 200, information included in the recognized QR code may be transmitted to the information reception unit 110. Alternatively, OCR recognition (i.e., text recognition) may be performed after scanning the prescription information by executing the platform or the application installed on the user terminal 200, and the prescription information inputted based on the OCR recognition may be transmitted to the information reception unit 110. Alternatively, according to an embodiment, the prescription information inputted by the user may be stored in a commercial cloud such as AWS or a separate server, in which case the prescription information may be transmitted to the information reception unit 110 via the cloud or the separate server.

Alternatively, according to an embodiment, a user's (i.e., patient's) prescription (i.e., prescription information) may be received via an EMR system or the like.

The information reception unit 110 may receive adverse reaction information from the user (S310).

The adverse reaction information may include condition information following drug administration, adverse reaction symptoms, effects and the like. Furthermore, the adverse reaction information may include any unexpected reaction following drug administration.

For example, the user (i.e., the patient) may take prescribed drugs based on the prescription information and then input symptoms related to the taken drugs (i.e., adverse reaction symptoms) through the platform or the application on the user terminal 200. These adverse reaction symptoms may then be transmitted to the information reception unit 110.

Alternatively, according to an embodiment, the adverse reaction information inputted by the user may be stored in a commercial cloud such as AWS or a separate server, in which case the adverse reaction information may be transmitted to the information reception unit 110 via the cloud or the separate server.

The analysis unit 120 may analyze the possibility of adverse reactions occurring based on drugs included in the prescription information and the adverse reaction information in the individualized adverse reaction database 150 to extract specific drugs that induce adverse reactions and determine alternative drugs (S320).

In this case, since the individualized adverse reaction database 150 may not collect sufficient individualized adverse reaction data at an early stage, the analysis unit 120 may further use external databases in addition to the individualized adverse reaction database 150. For example, as described above, external databases may include databases built by collecting, assessing and organizing pharmaceutical-related data from institutions such as the Ministry of Food and Drug Safety (MFDS), the Health Insurance Review and Assessment Service (HIRA), and the Ministry of Health and Welfare (MOHW). Alternatively, external databases may include databases established by agencies such as the MFDS, which collects and evaluates adverse reaction data.

In an embodiment, in analyzing the possibility of adverse reactions occurring, the analysis unit 120 may obtain information related to drugs and adverse reactions and analyze factors affecting the possibility of adverse reactions occurring according to drugs and adverse reaction symptoms based on the information, and provide the results of the analysis. For example, the analysis unit 120 may receive responses from users (e.g., user terminals 200, cloud servers storing responses, or separate servers) to questions related to the drugs included in the prescription information and adverse reaction data, and based on these responses, determine a degree of the possibility of adverse reactions occurring associated with the prescribed drugs. Subsequently, the analysis unit 120 may extract specific drugs from the prescription that are determined to have a possibility of adverse reactions based on the possibility of adverse reactions occurring.

As a specific example, the analysis unit 120 may determine whether adverse reaction symptoms reported by the patient a certain number of days after taking the prescribed drug are already included in the symptoms of the prescription initially registered by the patient. Alternatively, the analysis unit 120 may inquire whether the patient has previously experienced adverse reactions to the same or similar drugs. Additionally, the analysis unit 120 may determine whether the prescription includes contraindicated drug combinations and whether the observed adverse reactions correspond to those associated with contraindicated drug combinations. Furthermore, the analysis unit 120 may determine whether the adverse reaction occurred after the patient started taking the drug or if it existed beforehand. Moreover, the analysis unit 120 may determine whether there are any known adverse reactions associated with the drug the patient has taken. The analysis unit 120 may also determine whether the patient has any prior history for re-administration of the same drug. In other words, the analysis unit 120 may assess whether the adverse reaction experienced by the patient is likely related to the prescribed drug (the possibility of adverse reactions occurring) based on information regarding the patient's drug administration and adverse reaction symptoms.

Additionally, in an embodiment, when extracting a specific drug inducing an adverse reaction and determining an alternative drug based on the analysis of the possibility of adverse reactions occurring, the analysis unit 120 may analyze the possibility of adverse reactions occurring based on the drugs and the adverse reaction information included in the prescription information to extract the specific drug inducing the adverse reactions and determine an alternative drug by using a learning model trained based on the individualized adverse reaction database 150.

As a specific example, the analysis unit 120 may build the individualized adverse reaction database 150 using individualized adverse reaction data (e.g., user age, gender, symptoms, constitution, drug-adverse reaction correlation, possibility of adverse reactions occurring, individualized probability of adverse reaction occurring, etc.) obtained based on at least one of user information, prescription information, and adverse reaction information. The analysis unit 120 may generate a learning model for extracting AR-inducing drugs and determining alternative drugs through machine learning based on the individualized adverse reaction database 150. Subsequently, the analysis unit 120 may, based on the learning model, calculate the probability of adverse reactions for each drug by analyzing the possibility of adverse reaction occurring based on drugs within the prescription and adverse reaction symptoms, extract specific drugs inducing adverse reactions among drugs within the prescription and replace them with alternative drugs.

When, in the initial stage, there is insufficient data in the individualized adverse reaction database, the analysis unit 120 may, based on the learning model trained by further using external databases in addition to the individualized adverse reaction database 150, analyze the possibility of adverse reaction occurring based on drugs included in prescription information and adverse reaction information, extract specific drugs inducing adverse reactions among drugs within the prescription and replace the specific drugs with alternative drugs.

Additionally, in an embodiment, the analysis unit 120 may calculate the probability of adverse reactions by analyzing the possibility of adverse reactions occurring based on drugs included in prescription information and adverse reaction information according to statistical methodologies using the individualized adverse reaction database 150, extract specific drugs inducing adverse reactions based on whether the probability of adverse reactions exceeds a predefined threshold, and determine alternative drugs. For example, when a drug included in the prescription information has a probability of adverse reactions exceeding the predefined threshold, the analysis unit 120 may determine the drug as an adverse reaction-inducing drug and replace the drug with an alternative drug.

In cases where the individualized adverse reaction database is not sufficiently populated in the early stages, the analysis unit 120 may perform the statistical methodologies by using external databases in addition to the individualized adverse reaction database 150.

The information provision unit 130 may generate prescription information optimized based on the alternative drug and provide the prescription information to a user (S330).

That is, after detecting AR-inducing drugs among drugs within the prescription, the information provision unit 130 may provide optimized alternative drugs to replace the AR-inducing drugs to generate a revised prescription containing the alternative drugs. The user may receive optimized prescription information, including the alternative drugs, through the user terminal 200.

The update unit 140 may collect feedback information from users after drug administration based on the optimized prescription information and update the individualized adverse reaction database 150 accordingly (S340).

In an embodiment, after taking the prescribed drugs based on the optimized prescription information, users may input feedback information (e.g., post-administration symptoms) through the platform or the application of their user terminal 200. The update unit 140 may receive the inputted feedback information from the user terminal 200 and update the individualized adverse reaction database 150 based on the received feedback information. Alternatively, according to an embodiment, the feedback information inputted by the user may be stored in a commercial cloud such as AWS or a separate server, in which case the feedback information may be transmitted to the update unit 140 via the cloud or the separate server.

Since the individualized adverse reaction database 150 reflects the feedback information collected after drug administration based on the optimized prescription information that includes alternative drugs, it can improve the accuracy of individual adverse reaction data and maintain an optimized data state.

Accordingly, in some embodiments, the update unit 140 may perform learning to extract AR-inducing drugs and determine alternative drugs through machine learning based on the individualized adverse reaction database 150, which has been updated with the feedback information collected after drug administration based on the optimized prescription information. That is, by using a learning model trained to reflect the feedback information, it is possible to improve the accuracy of analysis and prediction in the process of extracting AR-inducing drugs and determining alternative drugs by analyzing the relationship between drug administration and the possibility of adverse reactions occurring. Here, machine learning may include various rule-based mechanisms for causality assessment.

Additionally, in some embodiments, the update unit 140 may detect various events (e.g., symptoms) that occur after drug administration to update the probability of adverse reactions. In this case, the update unit 140 may adjust the probability related to drug interaction risks using external databases (e.g., public DB built by collecting and organizing pharmaceutical-related data from institutions such as the Ministry of Food and Drug Safety (MFDS), Health Insurance Review & Assessment Service (HIRA), Ministry of Health and Welfare (MOHW), Korea Institute of Drug Safety & Risk Management (KIDS), or the like). Subsequently, a more accurate analysis of the relationship between drug administration and the possibility of adverse reactions occurring may be performed based on the adjusted probability, using the individualized adverse reaction database 150.

Furthermore, in some embodiments, the device 100 according to an embodiment of the present disclosure may provide at least one of the adverse reaction information, AR-inducing drugs, alternative drugs, optimized prescription information, or feedback information to the external server 300.

For example, the device 100 according to an embodiment of the present disclosure may provide information on AR-inducing drugs, alternative drugs, optimized prescription information, feedback information, or the like to an external server (300) (e.g., a hospital server or a pharmacy server). The external server 300 (e.g., a hospital server or a pharmacy server) may receive the above-mentioned patient-related information and utilize the patient-related information for diagnosis, prescription, dispensing, or usage instructions.

Alternatively, for example, the device 100 according to an embodiment of the present disclosure may provide optimized prescription information, including alternative drugs, to the external server 300 (e.g., a logistics server for delivery). The external server 300 (e.g., a logistics server for delivery) may use a delivery service company or a courier to deliver drugs dispensed based on the optimized prescription information to a corresponding patient.

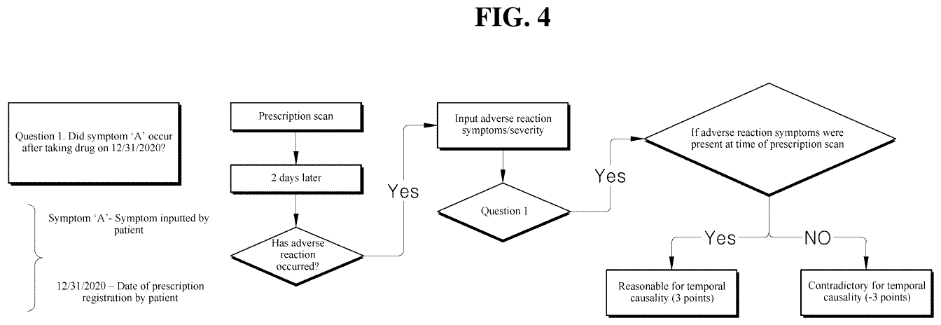

FIGS. 4 to 10 illustrate examples of algorithms for analyzing a causal relationship between drugs and adverse reactions or the possibility of adverse reactions occurring according to an embodiment of the present disclosure.

As described above, the analysis unit 120 may determine the causal relationship between drugs included in the prescription information and adverse reactions or the possibility of adverse reactions occurring based on responses to predefined questions after a user has taken a drug from the prescription information.

In an embodiment, referring to FIG. 4, the analysis unit 120 may determine the causal relationship between drugs and adverse reactions or assess the possibility of adverse reactions occurring by asking about the temporal relationship between drug administration and the occurrence of adverse events. For example, the analysis unit 120 may ask whether the adverse reaction symptom was included in the symptoms at the time the prescription was entered and calculate a causality score or degree of possibility of the adverse reaction occurring based on the response. As an example, all drugs prescribed on Dec. 31, 2020 should be separated by ingredient and then questions should be provided accordingly; if five drugs were prescribed and three drugs of them were in the previous prescription history, this would be considered contradictory for temporal causality (with a causality score of −3), while the other two drugs would be considered reasonable for temporal causality if they were not taken within 7 days of Dec. 31, 2020 (with a causality score of 3, or the degree of the possibility of adverse reactions occurring of 3).

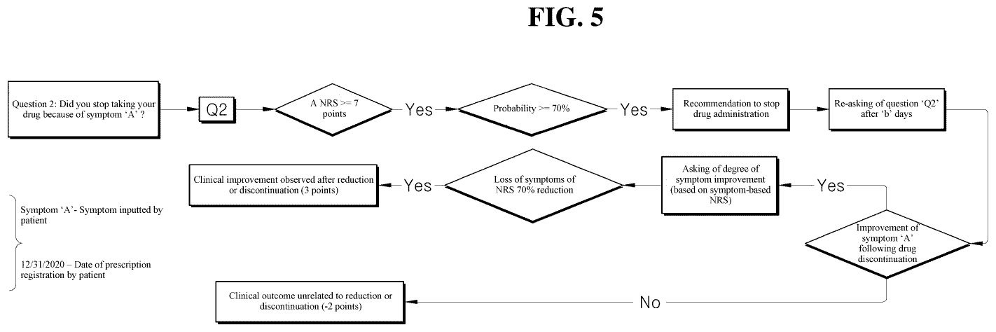

In an embodiment, referring to FIG. 5, the analysis unit 120 may ask for information about reducing or discontinuing the drug to determine a causal relationship between the drug and the adverse reactions or the possibility of the adverse reaction. For instance, the analysis unit 120 may ask whether a first adverse reaction has occurred a certain number of days after drug administration (denoted as “a” days), and if the adverse reaction symptoms are severe or there is the possibility of the adverse reactions, recommend discontinuation. Subsequently, the analysis unit 120 may ask for symptom improvement a certain number of days (“b” days) after discontinuation and based on the response, calculate the causality score or the degree of possibility of adverse reaction. For example, if symptom improvement is observed after drug reduction or discontinuation, the causality score or the degree of possibility of adverse reactions occurring may be calculated as 3, and if the clinical course is unrelated to drug reduction or discontinuation, the causality score or the degree of possibility of adverse reaction may be calculated as −2.

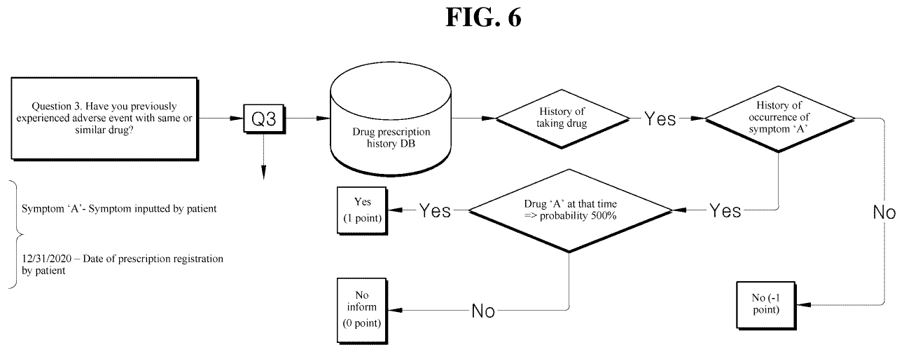

In addition, in an embodiment, referring to FIG. 6, the analysis unit 120 may determine a causal relationship between the drug and the adverse reaction or the possibility of the adverse reaction based on whether a patient has previously experienced an adverse reaction with the same or similar drug (i.e., adverse reaction history). For example, the analysis unit 120 may ask the patient if the same symptoms has occurred when exposed to the drug in the past, or query a database (e.g., an individualized adverse reaction database) and calculate a causality score or the degree of possibility of adverse reaction based on the results thereof. For example, if the patient has no history of adverse reactions to a corresponding drug, the causality score or the degree of possibility of adverse reaction may be calculated as −1. On the other hand, the causality score or the degree of possibility of the adverse reaction may be calculated as 1 if there is a history of occurrence of an adverse reaction symptom for the corresponding drug, and be calculated as 0 if there is no history of occurrence of the adverse reaction symptom.

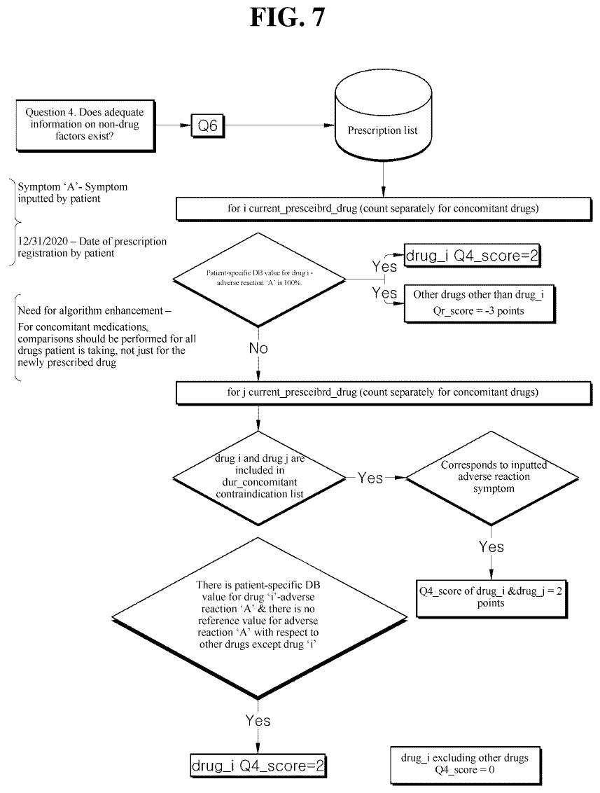

In addition, in an embodiment, referring to FIG. 7, the analysis unit 120 may ask if there is information about concomitant drugs to determine a causal relationship between the drug and the adverse reaction or the possibility of the adverse reaction. In determining the causal relationship or possibility of an adverse reaction with concomitant drugs, comparison may be performed for all of the drugs currently being taken by the patient, not just the newly prescribed drug. The analysis unit 120 may ask whether an adverse reaction symptom is present for each of the drugs included in the prescription list and whether an adverse reaction symptom is present for any drug other than a corresponding drug, and calculate a causality score or the degree of possibility of the adverse reaction based on the responses.

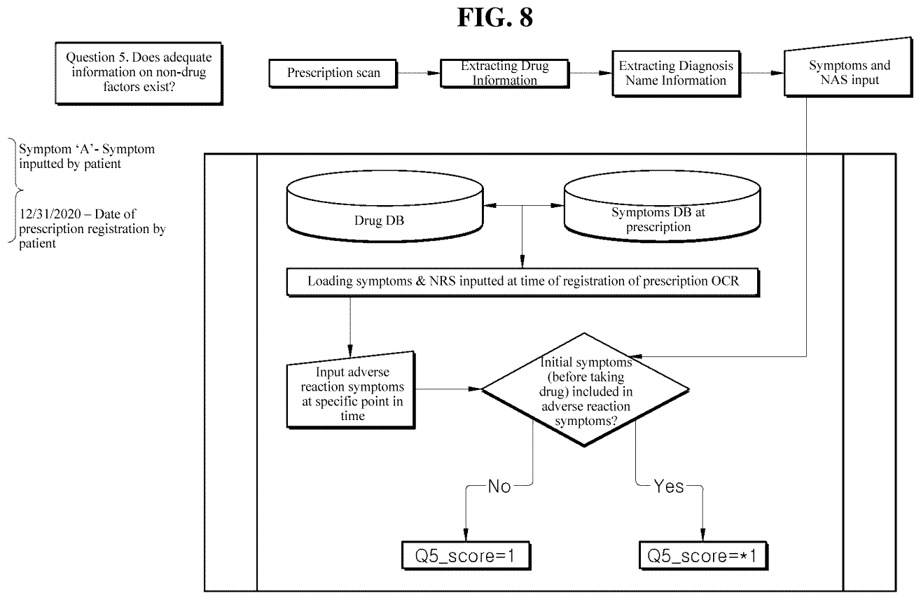

In addition, in an embodiment, referring to FIG. 8, the analysis unit 120 may ask whether there is information about non-drug factors to determine a causal relationship between the drug and the adverse reaction or the possibility of the adverse reaction occurring. For example, the analysis unit 120 may ask whether the symptoms of the adverse reaction include symptoms that were present when the prescription was registered and calculate a causality score or the degree of possibility of the adverse reaction based on the responses. In an example, the causality score or the degree of possibility of an adverse reaction occurring may be calculated as −1 if the adverse reaction symptom is included in the symptoms that were present when the prescription was registered, and the causality score or the degree of possibility of an adverse reaction occurring may be calculated as 1 if the adverse reaction symptom is not included in the symptoms that were present when the prescription was registered.

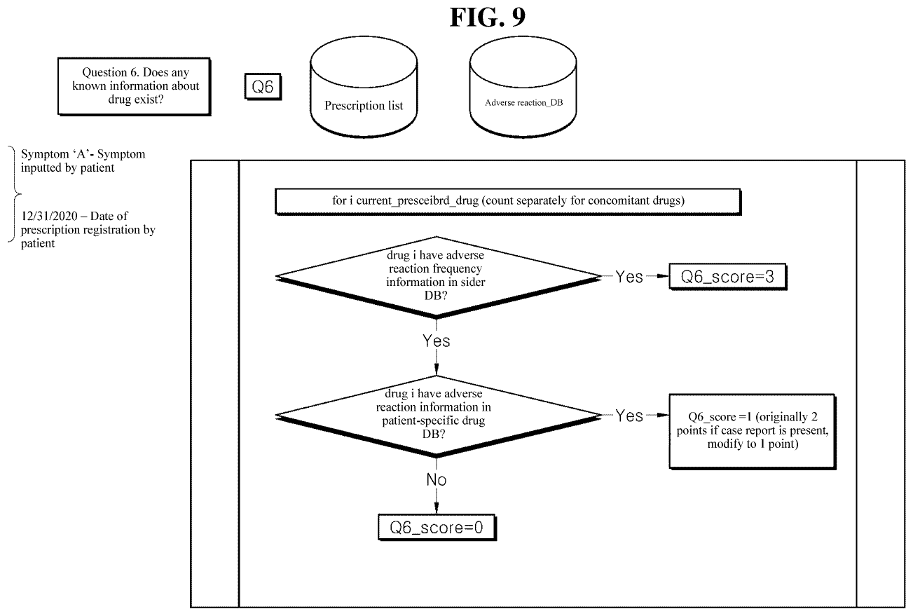

In addition, in an embodiment, referring to FIG. 9, the analysis unit 120 may determine a causal relationship between the drug and the adverse reaction or the possibility of the adverse reaction occurring by asking if any information is known about the drug. For example, the analysis unit 120 may query the database for the presence of adverse reaction frequency information for a corresponding drug and adverse reaction information that occurred in the patient, and calculate a causality score or a degree of probability of the adverse reaction occurring based on the results. For example, if there is the adverse reaction frequency information for the drug in the adverse reaction database, the causality score or the degree of possibility of the adverse event occurring may be calculated as 3. Alternatively, if there is no adverse reaction frequency information for the drug in the adverse reaction database, but there is adverse reaction information for the patient in the individualized adverse reaction database, the causality score or the degree of possibility of the adverse reaction occurring may be calculated as 1, and if there is no adverse reaction information for the patient, the causality score or the degree of probability of the adverse event occurring may be calculated as 0.

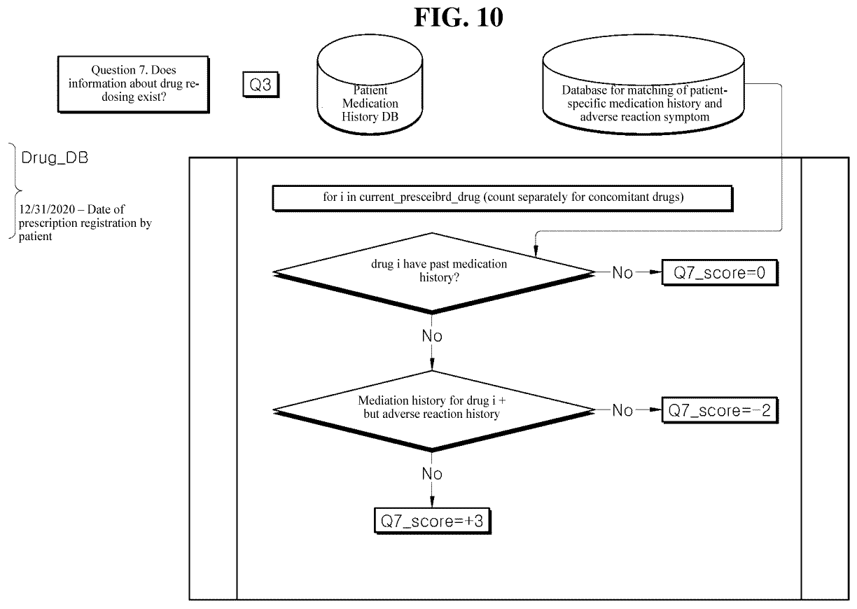

Also, in an embodiment, referring to FIG. 10, the analysis unit 120 may determine a causal relationship between a drug and an adverse reaction or the possibility of the adverse reaction occurring by asking if there is information about the re-dosing of the drug. For example, the analysis unit 120 may ask a patient whether he or she has a past history of taking a corresponding drug and whether he or she has a history of taking the drug and having an adverse reaction, or may query a database (e.g., a individualized adverse reaction database) and calculate a causality score or a degree of possibility of the adverse reaction based on the results. For example, if an individual has no past history of taking the corresponding drug, the causality score or the degree of possibility of an adverse reaction occurring may be calculated as 0. Alternatively, if the individual has a past history of taking the drug and a history of having an adverse reaction after taking the drug, the causality score or the degree of possibility of an adverse reaction occurring may be calculated as 3. Alternatively, if the individual has a past history of taking the drug and a history of having no adverse reaction after taking the drug, the causality score or the degree of possibility of an adverse reaction occurring may be calculated as −2.

In other words, a causality score or a degree of possibility of an adverse reaction occurring may be calculated based on the responses to the questions related to the causal relationship between the drug and the adverse reaction or the degree of possibility of the adverse reaction occurring as shown in FIGS. 4 to 10. The causality score or the degree of possibility of the adverse reaction occurring may be used to determine whether the adverse reaction experienced by the patient is causally related to drug administration or has a certain degree of possibility of the adverse reaction occurring, thereby extracting a specific drug inducing the adverse reaction from drugs included in the prescription information.

In an embodiment of the present disclosure, as an algorithm for analyzing the causal relationship between a drug and an adverse reaction or the probability of an adverse reaction occurring, the Korean causality assessment algorithm may be used along with the WHO-UMC and Naranzo scale, which are used by the Korea Institute of Drug Safety & Risk Management (KIDS) in the evaluation of drug adverse reaction relief.

According to the present disclosure as described above, it is possible to effectively detect AR-inducing drugs by individuals and provide optimized drugs by using a database (i.e., individualized drug adverse reaction database) that accumulates information such as efficacy and drug adverse reaction of drug administration by individuals.

Further, according to the present disclosure as described above, the accuracy and reliability of extracting AR-inducing drugs and determining alternative drugs may be improved by updating the database (i.e., the individualized drug adverse reaction database) by reflecting feedback information in the database after providing a prescription containing the optimized drug.

Furthermore, according to the present disclosure as described above, by configuring a causality determination algorithm or a ADR probability determination algorithm based on the results of answering various questions related to a causal relationship between a drug and a drug adverse reaction or a probability of a drug adverse reaction occurring, it is possible to easily determine whether a symptom occurring in a patient is causally related to a drug or is likely to occur as a drug adverse reaction.

In addition, according to the present disclosure as described above, it is possible to predict drug adverse reaction that will occur to a patient according to the type of drug when taking a drug through the analysis of the causal relationship between the drug and the drug adverse reaction or the possibility of the drug adverse reaction occurring, and prescribing and recommending a drug with a low risk of occurrence of drug adverse reactions.

The method according to the present disclosure described above may be implemented as a program (or application) to be executed in combination with a computer, which is hardware, and stored in a medium.

On the other hand, the disclosed embodiments may be implemented in the form of a recording medium storing instructions executable by a computer. The instructions may be stored in the form of program codes, which, when executed by the processor, may generate program modules to perform the operations of the disclosed embodiments. The recording medium may be implemented as a computer-readable recording medium.

The computer-readable recording medium includes all types of recording media that store instructions that can be deciphered by a computer. For example, the computer-readable recording medium may include read only memory (ROM), random access memory (RAM), magnetic tape, magnetic disc, flash memory, optical data storage, and the like.

The embodiments of the disclosure have been described above with reference to the accompanying drawings. A person skilled in the art to which this disclosure pertains will understand that the present disclosure may be practiced in forms different from the disclosed embodiments without changing the technical idea or essential features of the present disclosure. The disclosed embodiments are illustrative and should not be construed as limiting.

According to the the embodiments of the present disclosure, it is possible to effectively detect AR-inducing drugs by individuals, prevent the occurrence of adverse reactions and provide optimized drugs by using a database (i.e., an individualized adverse reaction database) that accumulates information such as efficacy and drug adverse reaction of drug administration by individuals.

Further, according to the embodiments of the present disclosure, by updating a database (i.e., an individualized adverse reaction database or an individualized database for possibility of an adverse reaction occurring) to reflect feedback information on whether the adverse reaction has occurred after taking the drug and symptoms, after providing a prescription containing an optimized drug, it is possible to improve accuracy and reliability in extracting AR-inducing drugs and determining alternative drugs.

Furthermore, according to the embodiments of the present disclosure, by configuring a causality determination algorithm based on the results of answering various questions related to a causal relationship between a drug and a drug adverse reaction or a probability of a drug adverse reaction occurring, it is possible to easily determine whether a symptom occurring in a patient is causally related to a drug or is likely to occur as a drug adverse reaction.

In addition, according to the embodiments of the present disclosure, it is possible to predict drug adverse reaction that will occur to a patient according to the type of drug when taking a drug through the analysis of the causal relationship between the drug and the drug adverse reaction or the possibility of the drug adverse reaction occurring, and prescribing and recommending a drug with a low risk of occurrence of drug adverse reactions.

However, effects of the present disclosure are may not be limited to the above-described effects. Although not described herein, other effects of the inventive concept can be clearly understood by those skilled in the art from the following description.

While the present disclosure has been described with reference to embodiments, it will be apparent to those skilled in the art that various changes and modifications may be made without departing from the spirit and scope of the present disclosure. Therefore, it should be understood that the above embodiments are not limiting, but illustrative.

What is claimed is:

1. A method performed by a server for detecting an adverse reaction-inducing drug and providing an optimized drug using individualized adverse reaction data, the method comprising:
receiving prescription information from a user;
receiving adverse reaction information from the user;
extracting a specific drug inducing an adverse reaction by analyzing a possibility of the adverse reaction occurring based on drugs included in the prescription information and the adverse reaction information using an individualized adverse reaction database and determining an alternative drug;
generating optimized prescription information based on the alternative drug and providing the optimized prescription information to the user; and
collecting feedback information after drug administration based on the optimized prescription information from the user to update the individualized adverse reaction database.

2. The method of claim 1, wherein the extracting of the specific drug inducing the adverse reaction and determining of the alternative drug includes extracting the specific drug inducing the adverse reaction by analyzing the possibility of the adverse reaction occurring based on the drugs included in the prescription information and the adverse reaction information and determining an alternative drug, using a learning model trained based on the individualized adverse reaction database.

3. The method of claim 1, wherein the extracting of the specific drug inducing the adverse reaction and determining of the alternative drug includes:
calculating a probability of the adverse reaction by analyzing the possibility of the adverse reaction occurring based on the drugs included in the prescription information and the adverse reaction information using the individualized adverse reaction database according to a statistical methodology; and
extracting the specific drug inducing the adverse reaction and determining the alternative drug based on whether the possibility of the adverse reaction occurring exceeds a predetermined threshold.

4. The method of claim 1, wherein the extracting of the specific drug inducing the adverse reaction and determining of the alternative drug includes:
receiving a response from the user to a question relating to the possibility of the adverse reaction occurring based on the drugs included in the prescription information and the adverse reaction information; and
determining, based on the response, the possibility of the adverse reaction occurring based on the drugs included in the prescription information and the adverse reaction information.

5. The method of claim 2, further comprising:
building the individualized adverse reaction database using individualized adverse reaction data obtained based on at least one of user information, the prescription information, and the adverse reaction information; and
generating, the learning model for extracting an adverse reaction-inducing drug and determining an alternative drug through machine learning based on the individualized adverse reaction database.

6. The method of claim 5, wherein the learning model is further trained to extract the adverse reaction-inducing drug and determine the alternative drug through the machine learning based on the individualized adverse reaction database updated with feedback information collected after drug administration based on the optimized prescription information.

7. The method of claim 1, wherein the extracting of the specific drug inducing the adverse reaction and determining of the alternative drug includes extracting the specific drug inducing the adverse reaction and determining the alternative drug by further using the individualized adverse reaction database and external databases.

8. The method of claim 1, further comprising:
providing at least one of the adverse reaction information, the specific drug inducing the adverse reaction, the alternative drug, the optimized prescription information, or the feedback information to an external server.

9. A computer program stored on a computer-readable recording medium, coupled to a computer that is hardware, for performing the method of claim 1.

10. A device for providing a method for detecting an adverse reaction-inducing drug and providing an optimized drug using individualized adverse reaction data, the device comprising:
an information reception unit configured to receive prescription information from a user and receive adverse reaction information from the user;
an analysis unit configured to extract a specific drug inducing an adverse reaction by analyzing a possibility of the adverse reaction occurring based on drugs included in the prescription information and the adverse reaction information using an individualized adverse reaction database and determine an alternative drug;
an information provision unit configured to generate optimized prescription information based on the alternative drug and provide the optimized prescription information to the user; and
an update unit configured to collect feedback information after drug administration based on the optimized prescription information from the user to update the individualized adverse reaction database.
